(19) United States
(12) Patent Application Publication
(10) Pub. No.: US 2010/0152580 A1
(43) Pub. Date: Jun. 17, 2010

(54) LUNG DIAGNOSIS APPARATUS WITH FOUR ULTRASOUND ELEMENTS

(76) Inventor: Peter Ganshorn, Munnerstadt (DE)

Correspondence Address: EDWIN D. SCHINDLER, FIVE HIRSCH AVENUE, P.O. BOX 966, CORAM, NY, 11727-0966, US

(21) Appl. No.: 12/634,941

(22) Filed: Dec. 10, 2009

(30) Foreign Application Priority Data
Dec. 17, 2008 (DE) 10 2008 063 503.0

(51) Int. Cl. A61B 8/00; A61B 5/097; A61B 5/087

(52) U.S. Cl. 600/437; 600/543; 600/538

(57) ABSTRACT

A lung diagnosis apparatus for measuring the flow, the flow rate, the respiratory air density and the molar mass of a living organism, includes a respiration tube through which respiratory air flows. On the outer surface of the respiration tube, on mutually opposite sides thereof, there are mounted two tube nozzles, of which the longitudinal axis thereof, or the nozzle axis, extends in an inclined manner relative to the longitudinal axis of the respiration tube, or tube axis. In the two tube nozzles, either a first ultrasound transmitter or a first ultrasound receiver is fixed. An electronic module, which actuates the first ultrasound transmitter and the first ultrasound receiver, evaluates their signals. A third tube nozzle and a fourth tube nozzle are arranged on mutually opposite sides of the respiration tube. The longitudinal axes of the third tube nozzle and the fourth tube nozzle define cross-nozzle axes, and extend orthogonally to the tube axis. In the third tube nozzle and the fourth tube nozzle, either a second ultrasound transmitter or a second ultrasound receiver are fixed.

The invention relates to a lung diagnosis device for measuring the flow rate, the flow, the respiratory air density D and the molar mass of the respiratory air of a living organism, comprising a respiration tube, through which the respiratory air flows, and on the outer surface, on mutually opposite sides, of which there are mounted two tube nozzles, of which the longitudinal axis, the nozzle axis, extends inclined with respect to the longitudinal axis of the respiration tube, the tube axis, by an angle, and in which a first ultrasound transmitter or a first ultrasound receiver is fixed, and an electronic module, which actuates the ultrasound transmitter and the ultrasound receiver and evaluates their signals.

For the diagnosis of different lung illnesses, it is necessary to determine the flow rate, the flow, the respiratory air density and the molar mass of the respiratory air, Various methods and various apparatus are known for this.

In the prior art, European Patent EP 0 653 919, Harnoncourt, describes a measurement zone, through which respiratory air flows and on which an ultrasound transmitter-receiver pair is arranged obliquely to the longitudinal axis of a measurement tube. With these two cells, the travel time of an ultrasound pulse through the flowing respiratory gas is measured and, from this, the flow rate is calculated.

For measurement of the molar mass, one temperature sensor in each case is disposed at the inlet to and outlet from the measurement zone. From these two measurement values for the gas temperature, the temperature in the region of the ultrasound measurement zone is assumed as an average value. From the travel time measurements, the respective sound velocity is determined.

From the two values, the respective molar mass is calculated.

A significant disadvantage of this measurement method is that the principle of a further increase of the measurement accuracy prevents the exact temperature of the gas in the ultrasound measurement zone from being known, but it must be assumed as an average value between the values measured at the inlet and outlet.

A further, even more serious disadvantage in the measurement of the molar mass and the density is caused by the fact that the ultrasound transmitter and the ultrasound receiver must be installed in tube nozzles that are arranged obliquely on the outer surface. By virtue of its inclined arrangement with respect to the longitudinal axis of the respiration tube and by virtue of a corresponding length of the nozzle, the ultrasound transmitter and the ultrasound receiver are prevented from projecting into the respiration tube and there restricting the flow of the respiratory air, thereby falsifying measurements of flow and flow velocity.

Their disadvantage is that respiratory air that has penetrated into the tube nozzle becomes turbulent there, so that the even flow of respiration is interrupted, thus restricting the accuracy of the measurement of molar mass and density. Even if close-meshed grilles are arranged between the tube nozzle and interior of the respiration tube, which reduce the penetration of respiratory air into the tube nozzle, but do not restrict the ultrasound in the measurement zone too much, the inaccuracy remains, since, with this arrangement, there is still turbulence of the respiratory air in the tube nozzle which limits the measurement accuracy.

Against this background, it is the object of the invention to develop a lung diagnosis apparatus that permits the measurement of molar mass and density of the respiratory air in a range of laminar flow of the respiratory air without turbulence and free of dead spaces, and with which the flow rate and flow are measurable, and which can be easily expanded for measuring the average value of flow and molar mass over a respiratory cycle and for determining the anatomic dead space of a respiratory system of a living organism.

As a solution, the invention presents the fact that a third and a fourth nozzle are arranged on mutually opposite sides of the respiration tube, the longitudinal axis of which—the cross-nozzle axis—extends orthogonally to the tube axis and, in the nozzles, there are fixed a second ultrasound transmitter or a second ultrasound receiver in each case.

The gist of the invention is thus that the measurement of flow rate and flow is transferred to a first ultrasound measurement zone, which extends diagonally to the longitudinal axis of the tube and the measurement of respiratory air density and molar mass is trusted to a second ultrasound measurement zone, which operates not diagonally to the air stream but transversely to the air stream, and thereby successfully excludes several error sources of the diagonal measurement zone.

By elimination of the tube nozzles, the air turbulence occurring there is also eliminated, and thereby the inhomogeneities of density caused thereby. Furthermore, the temperature drop caused by the turbulence in the tube nozzles is also eliminated, which is further increased by the moisture of the respiratory air. A further disadvantage is that, as a result of the measurement zone oriented transversely to the flow direction of the air, no temperature gradient occurs in the measurement zone, which would need to be compensated, and the temperature sensors that are otherwise necessary are also eliminated. By this means the achievable measurement accuracy is significantly increased as a matter of principle.

In a further distinct embodiment, the invention refers to measurements that require both the flow velocities or the flow (F) as well as the respiratory air density or the molar mass of the respiratory air. For such measurement tasks, the invention proposes that the cross-nozzle axis intersects the nozzle axis, that is to say that the centre of the first ultrasound measurement zone extending diagonally to the air stream intersects with the second ultrasound measurement zone extending transversely to the air stream. By this means it is achieved that the measurement values from both ultrasound measurement zones register the same air volume in the same time instant, which significantly increases the accuracy of the parameters calculated therefrom.

In an even further optimized arrangement, the angle between the tube axis and the nozzle axis is so small that the distance between the two tube nozzles on the outer surface—measured in the direction of the tube axis—is at least as large as the outer diameter of a nozzle. It thereby becomes possible to fit the two nozzles for the second ultrasound measurement zone between the two diagonal tube nozzles of the first ultrasound measurement zone.

Then the nozzle axis and the cross-nozzle axis can be arranged in a common plane, which allows measurement errors from different measurement planes of the two ultrasound measurement zones to be eliminated. A further advantage of the arrangement is that the connections of the ultrasound transmitter and receiver lie in one plane and can thereby be produced with reduced outlay.

As the next stage of opitmization, the invention proposes that the profile of the respiration tube in the region of the tube nozzles and nozzles is a plane. By this means it is achieved principally for the nozzles that the plane surface of the ultrasound elements of the second measurement zone terminate flush with the inside of the respiration tube, so that the transition from the flat wall of the respiration tube onto the surface of the ultrasound element permits unrestricted continuation of a laminar flow through the second ultrasound measurement zone, and the generation of further turbulence is avoided.

In a further alternative embodiment, an ultrasound transmitter can also operate as an ultrasound receiver and vice versa. By this means, it is achieved that the measurement zone can be measured alternately in one direction and then in the other. By this means, direction-dependent effects or disturbances in an averaging are necessarily compensated.

In a further variant, an optimization of the settling time of the ultrasound elements is proposed. In particular for the second, relatively short ultrasound measurement zone, which measures transversely to the air stream, with a conventional inner diameter of the respiration tube of about 20 mm and a frequency of the ultrasound measurement element of 300 to 400 kHz, travel time is about 50 to 60 μseconds. For the change between transmission and reception, in this configuration, for a time interval of the individual measurements of about 1 millisecond, the settling time is already relatively short.

The invention therefore proposes that an ultrasound transmitter can be more rapidly set to the passive state after deactivation by actuation with an alternating voltage that is inverse with respect to its natural resonance oscillation. It is thus not only waited until the oscillations of the ultrasound have decayed, but by means of a phase-synchronized but opposite-direction energy supply, that the oscillation decays even faster. That is particularly advantageous if the ultrasound transmitter has emitted a sound pulse and then is to become active again as receiver for the sound pulse of the second ultrasound element disposed opposite it in the respective measurement zone.

If the measurement frequency lies in the range of 1,000 Hz, there remains for each cycle time a total time of one millisecond, so that a “time gain” of several μseconds already becomes advantageously noticeable.

In the prior art, for all diagnostic parameters, which include both the molar mass and flow, the measurement accuracy was limited by the fact that the ratio of molar mass to flow fluctuates somewhat with non-uniform, intermittent exhalation. Therefore the average value from the start of the exhalation of the air from the alveolae, the so-called alveolar air, until the end of exhalation should be determined.

However, this maximally extended averaging is not possible in the prior art, since it cannot be determined with sufficient accuracy and with sufficient rapidity, in which the exhaled air no longer comes from the dead space of the respiratory system but from the alveolar air.

An especially high measurement frequency is thus necessary if, for an improvement of the diagnostic quality, the measurement values for density and molar mass are not only to be registered at any arbitrary point of a respiratory cycle, but are to be determined beyond one breath, specifically only within the exhalation of the alveolar air, that is to say the air from the alveolae.

For the purposes of averaging, the region of dead-space air, i.e. the air from the pharyngeal cavity, airways and bronchi, is excluded. For this purpose, it is necessary to precisely determine the dead-space end point, that is to say the time at which the exhaled air no longer comes from the anatomical dead space but from the alveolae.

As is known, the transition from dead-space air to alveolar air is marked by the dramatic drop of the curve of respiratory air density over the time within about 100 milliseconds, to a value that is approximately constant for a short time. The technical measurement of this process is made more difficult by the fact that the constant value is also only held for about 100 milliseconds and, within this time, under very accurate analysis, is only to be described as “constant” in so far as it fluctuates about a constant average value with relatively low deviations.

For an accurate registration of the time at which the decreasing range merges into the approximately constant range, both ranges must be replaced by a straight line in each case, the intersection point of which is then the exact point in time of the transition. It is readily apparent, that this requires multiple measurements within the two regions.

As mentioned, for an adult human, the respiratory air density decreases within about 100 milliseconds and then fluctuates about the approximately constant value for a further 100 milliseconds. If, e.g. measurements are performed with a measurement frequency of 1 kilohertz, measurement points occur at intervals of one millisecond. These measurement values must be stored in a memory. The electronic module of a lung diagnosis apparatus according to the invention is structured such that the dead-space end point can be very precisely measured in a module appropriate for this purpose, by the following three steps:

In the first step, the lowest value from the flow of breath and a preselectable number of the next low values are selected, and, in the time range covered thereby, the average value of all the measurements is formed and evaluated as a constant value.

In the second step, from the range from the start of exhalation until the approximately constant value is reached for the first time, a central portion of the curve with a preselectable width is selected, and, from all measurement values of this range, a straight line with a particular gradient is determined.

In the third step, the intersection of these straight lines with the value determined for the constant value is evaluated as the time point for the dead-space end point.

With the currently available electronic storage modules and microprocessors, that is readily possible.

In this manner, the exact start of exhalation of alveolar air is marked. The end of this phase is marked with completely adequate precision in that the flow of respiratory air falls to zero.

Since a lung diagnosis apparatus according to the invention in this embodiment permits the exact determination of the dead-space end point, at which the exhaled air no longer comes from the anatomical dead space but from the alveolae, the determination of the anatomical dead space itself is possible. To this end, the flow and the respiratory air density of the respiratory air during exhalation must be measured continuously and simultaneously over time, and the integral of the flow from the start of exhalation until the dead-space end point must be formed. Here, the start of exhalation is to be registered with completely satisfactory accuracy by the transition of the flow velocity from zero to a measurable value.

Since, in the measurement, the measurement chamber volume of the measurement zone is always also registered, too, this volume must be subtracted from the measurement result. Since the interior volume of the measurement zone is very accurately known, and since the flow within the measurement zone is also approximately laminar, the measurement space volume can be directly subtracted from the measurement result.

If, for example, the ultrasound elements are arranged in the centre of the measurement zone, the anatomical dead space emerges as the total of the measured volume from the start of exhalation to the dead-space end point, reduced by half the measurement space volume. or as a formula

$$Vat = {\sum }_{{T}_{0}}^{η} Vg - \frac{1}{2} Vap$$

### Where

Vat is the anatomical dead-space volume
Vg is the breath volume measured in the measurement zone
Vap is the measurement space volume of the measurement zone
T₀ is the start of exhalation, and
Tt is the dead-space end point, which marks the end of the time from the start T₀ of exhalation until the complete expulsion of the air inhaled into the anatomical dead space (Vat).

In the prior art, the known lung diagnosis apparatus is not suitable for the diagnosis of the breathing system of small children or of small animals, since the stream of respiratory air is very weak and non-uniform, and lacks any possibility to amplify and stabilise the breath stream by means of a suitable breath command. However, the increased measurement velocity and the increased measurement accuracy of the lung diagnosis apparatus according to the invention make such applications possible, now.

As a further embodiment that will probably be used frequently in practice, the invention proposes that a second tube is inserted into the respiration tube, which has an opening in each case in the region of the tube nozzle and the nozzle, which are covered with a sound-transmitting material. In the interests of perfect hygiene, this tube is suitable for disposal after once-only use, and can be replaced by a new sterile second tube.

This tube thus has openings in the region of the four ultrasound elements. For the purpose of hygienic separation of the ultrasound elements of the breath stream (A) of the respective apparatus user, patients should cover these openings with a material that is transmissible to ultrasound but, to the greatest extent, keeps moisture, suspended particles and air away from the nozzle and the ultrasound elements.

Further details and features of the invention are explained below in greater detail with reference to an example. The illustrated example is not intended to restrict the invention, but only to explain it. In schematic view,

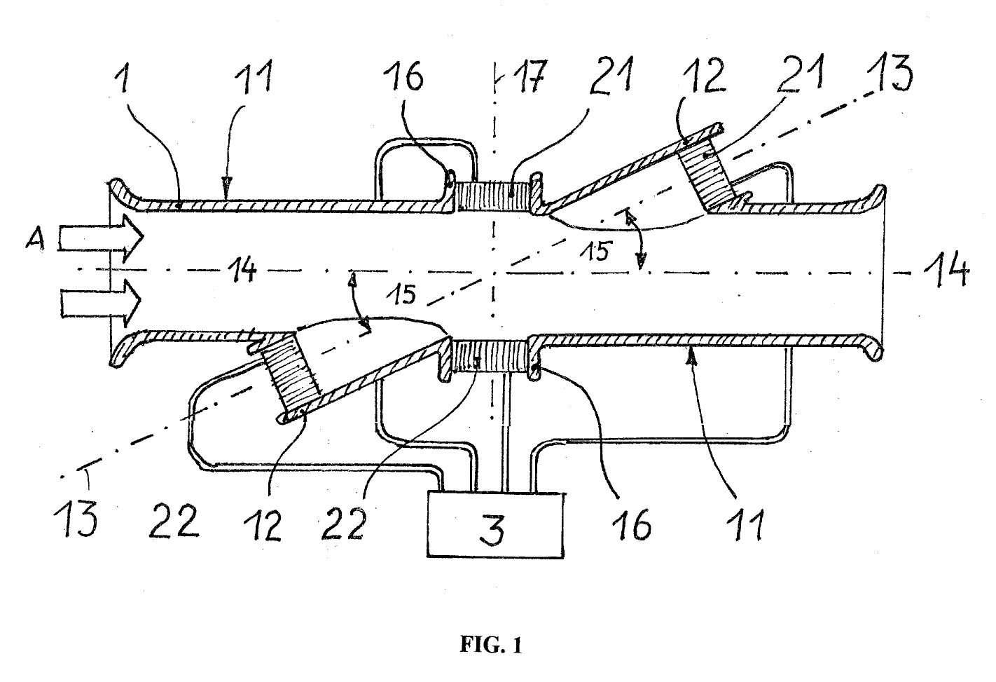

FIG. 1 shows a longitudinal section through a lung diagnosis apparatus

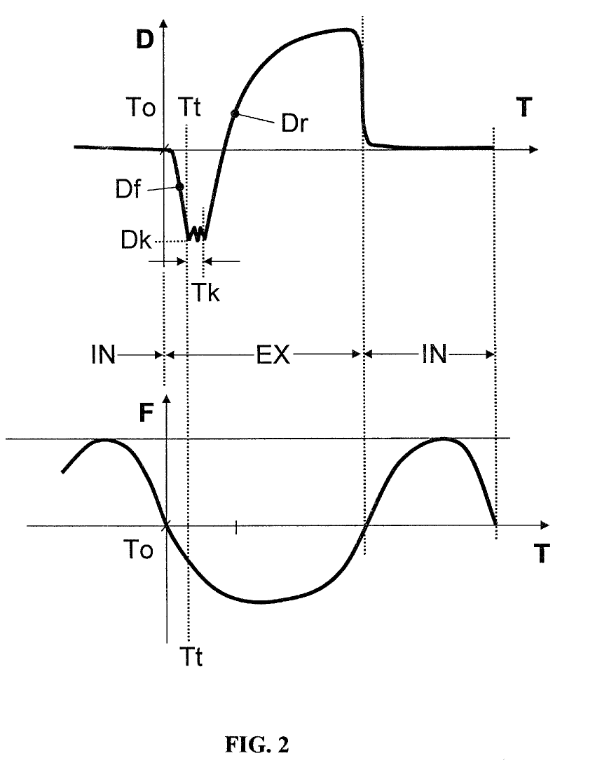

FIG. 2 shows schematic variations of the respiratory air density and the flow

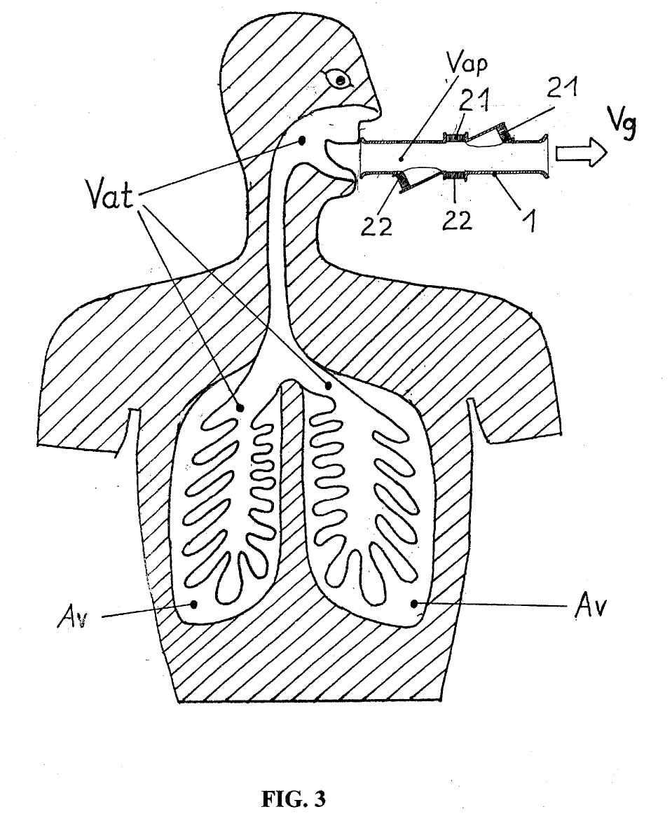

FIG. 3 shows a section through a patient with a lung diagnosis apparatus.

FIG. 1 shows the longitudinal section through a lung diagnosis unit according to the invention. Two double arrows symbolize the respiratory air (A), which streams in at the left end of the respiration tube (1), streams along the tube axis (14) and emerges again at the right end.

On the outer surface (11) of the respiration tube (1), there are mounted two obliquely disposed tube nozzles (12) for the first ultrasound measurement zone (21, 22). FIG. 1 makes it clear that the tube nozzles (12) are dimensioned long enough that the two ultrasound elements (21, 22) do not project into the cross-section of the respiration tube (1). This results in a relatively long measurement zone along the nozzle axis (13) between an ultrasound transmitter (21) and an ultrasound receiver (22).

The smaller the angle (15) between the nozzle axis (13) and the tube axis (14), the longer is the measurement zone. A further advantage of a possibly small angle (15) is that, on the outer surface (11), the distance between the two nozzles (12), projected onto the tube axis, becomes increasingly larger.

Depending on the diameter of the respiration tube (1) and the diameter of the nozzles (2), the angle (15) must not exceed a specific value in order to offer sufficient space on the outer surface (11) for the third and fourth nozzles (16), which receive the ultrasound transmitter (21) and the ultrasound receiver (22) for a second measurement zone, which extends along the cross-nozzle axis (17).

In FIG. 1, it quickly becomes clear that the cross-nozzle axis (17) extends orthogonally to the tube axis (14) and thereby transversely to the stream of respiratory air (A). It is also very clear that this measurement zone is absolutely uninfluenced by turbulence and streams that may form in the—approximately triangular in FIG. 1—interior space of the two tube nozzles (12).

It is also very clear that the first measurement zone along the nozzle axis (13) and the second measurement zone along the cross-nozzle axis (17) intersect at a common point on the tube axis (14). Both measurement zones thus register the same volume of air at the same time. By this means, this embodiment eliminates measurement errors from different positions of the measurement and at different breathing air velocities and at different breathing air temperatures.

In FIG. 1, it is shown schematically that both ultrasound transmitters (21) and both ultrasound receivers (22) are connected to an electronic module (3), which actuates all ultrasound transmitters and receivers (21, 22) and evaluates their signals.

In addition, the electronic module (3) in the corresponding embodiments also contains the structures for calculating further diagnostically very important values, as is explained in the following two figures.

In FIG. 2, the upper curve of the schematic variation of the breathing air density (D) over a cycle consisting of exhalation (EX) and inhalation (IN). In the lower portion, on the same time axis, the variation of flow (F) is plotted.

In the upper curve, the respiratory air density (D), it can be clearly seen that, with the start of exhalation (EX), the respiratory air density (D) drops off steeply with the falling flank (Of) until it reaches the approximately constant value (Dk). For the short time (Tk), the respiratory air density (D) fluctuates approximately about the constant value (Dk) and then merges into the rising flank (Dr). With the end of exhalation (EX) and the start of inhalation (IN), the respiratory air density (D) fails suddenly to zero again.

In the upper curve of FIG. 2, it can be clearly seen that the falling flank (Df) can be approximated by a straight line with limited computational outlay.

Likewise, FIG. 2 shows that the fluctuations about the constant value (Dk) can be combined with high precision in a single average value (Dk). If this average value (Dk) is plotted as a straight line—parallel to the time axis, it quickly becomes plausible in FIG. 2 that the intersection of these straight lines, with the falling flank (Df) of the variation of the respiratory air density, which has also been replaced by a straight line, reproduces the dead-space end point (Tt) with relatively high accuracy.

The lower curve, the flow (F), over time (T), demonstrates the plausibility that, together with the—known—volume (Vap) of the respiration tube (1), the dead-space volume (Vat) can be exactly calculated.

FIG. 3 shows a cross-section through a lung diagnosis apparatus according to the invention and a patient using the apparatus. The patient is drawn greatly simplified and stylized as a torso, in which the respiratory tract and its subdivision into the anatomic dead space (Vat) and the alveolar space (Av) are symbolically represented.

The part of the respiratory tract from the mouth space to the bronchia is the anatomic dead space volume (Vat), in which no gas is exchanged from the respiratory air with the blood. Following this, within the lung, the bronchia are surrounded by the alveolae (Av), which in FIG. 1 are not drawn individually but are represented by the area between symbolically sketched bronchia in the interior and the outline of the lung.

FIG. 1 shows how a lung diagnosis apparatus is applied to the patient's mouth. It contains two measurement zones (21, 22), of which one extends at an angle (15) to the direction of the respiratory air (A) and serves for the measurement of the flow velocity and flow (F), and the other extends perpendicular to the stream of respiratory air (A) and serves for measurement of the respiratory air density (D) and molar mass.

In FIG. 3, the entire volume (Vg) measured in the measurement zone is represented by a double arrow before the respiration tube (1). It is very clear that the entire measured volume (Vg) is the total of the anatomical dead-space volume (Vat) and the alveolar volume (Av) and the front portion of the measurement space volume (yap) in the respiration tube (1) from the mouthpiece to the measurement zones.

The electronic module (3)—which is not drawn here—can calculate the anatomical dead space volume (Vat) from the stored measurement values by forming the integral of the measured volume (Vg) from the time (T₀) until the dead-space end point (Tt) and from it subtracting the front portion of the known measurement chamber volume (Vap).

In FIG. 3, it also very quickly becomes clear that, to use the apparatus, the patient can breath virtually unrestricted through the breathing tube (1).

### LIST OF REFERENCE CHARACTERS

A Respiratory air of a living organism
Av Alveolae, gas-exchanging portion of the respiratory system
D Respiratory air density
Df Falling respiratory air density after the beginning of exhalation (EX)
Dk Constant value of respiratory air density
Dr Rising flank of the variation of the respiratory air density
EX Exhalation
F Flow, velocity of the respiratory air
IN Inhalation
T Time
Tk Short time during which the respiratory air density remains approximately at the value Dk
T₀ Start of exhalation (EX)
Tt Dead-space end point, time from the beginning T_{D} of exhalation until the complete expulsion of the air inhaled into the anatomical dead space (Vat).
Vap Air volume in the diagnosis apparatus
Vat Anatomical dead space, does not exchange gas with the blood
Vg Total measured air volume
1 Respiratory air through which the respiratory air A flows
11 Outer surface of the respiration tube 1
12 Tube nozzle for the first ultrasound measurement zone 21, 22
13 Nozzle axis, longitudinal axis of a tube nozzle 12
14 Tube axis, longitudinal axis of the respiration tube
15 Angle between the nozzle axis 13 and tube axis 14
16 Nozzle for second ultrasound measurement zone 21, 22
17 Cross-nozzle axis of the nozzle 16
21 Ultrasound transmitter in tube nozzle 12 or in nozzle 16
22 Ultrasound receiver in tube nozzle 12 or in nozzle 16
3 Electronic module, actuates the ultrasonic transmitter 21 and the ultrasonic receiver 22 and evaluates their signals.

What is claimed is:

1. A lung diagnosis apparatus for measuring flow, flow rate, respiratory air density and the molar mass of respiratory air of a living organism, comprising:
a respiration tube through which respiratory air is flowable having an outer surface on which there are mounted on mutually opposite sides of the outer surface a first tube nozzle and a second tube nozzle, said first tube nozzle and said second tube nozzle having a longitudinal axis extending at an incline relative to a longitudinal axis of said respiration tube, and defining an angle formed between the longitudinal axis of said respiration tube and the longitudinal axis of said first tube nozzle and said second tube nozzle, said respiration tube further including a third tube nozzle and a fourth tube nozzle on opposite sides of said respiration tube having a longitudinal axis defining a cross-nozzle axis extending orthogonally to said longitudinal axis of said respiration tube;
a first ultrasound transmitter fixed in said first tube nozzle;
a first ultrasound receiver fixed in said second tube nozzle;
a second ultrasound transmitter fixed in said third tube nozzle;
a second ultrasound receiver fixed in said fourth tube nozzle; and,
an electronic module for activating said first ultrasound transmitter, said first ultrasound receiver, said second ultrasound transmitter and said second ultrasound receiver and for evaluating signals thereof.

2. The lung diagnosis apparatus according to claim 1, wherein said third tube nozzle and said fourth tube nozzle, relative to the longitudinal axis of said respiration tube, are between said first tube nozzle and said second tube nozzle with the cross-nozzle axis crossing the longitudinal axis of said first tube nozzle and said second tube nozzle.

3. The lung diagnosis apparatus according to claim 2, wherein the angle formed between the longitudinal axis of said respiration tube and the longitudinal axis of said first tube nozzle and said second tube nozzle is sufficiently small so that the distance between said first tube nozzle and said second tube nozzle on the outer surface of said respiration tube, as measured in a direction of the longitudinal axis of said respiration tube, is greater than an outer diameter of at least one of said third tube nozzle and said fourth tube nozzle.

4. The lung diagnosis apparatus according to claim 3, wherein the longitudinal axis of said first tube nozzle and said second tube nozzle and the cross-nozzle axis are in one plane.

5. The lung diagnosis apparatus according to claim 1, wherein said respiration tube as a profile in a region of said first tube nozzle and said second tube nozzle vis-à-vis said third tube nozzle and said fourth tube nozzle that is planar.

6. The lung diagnosis apparatus according to claim 1, wherein said second ultra-sound transmitter has a surface facing said respiration tube and said second ultrasound receiver has a surface that terminates flush with an inner wall of said respiration tube.

7. The lung diagnosis apparatus according to claim 1, wherein said electronic module is capable of measuring a flow rate and a flow of respiratory air via said first ultrasound transmitter and said first ultrasound receiver.

8. The lung diagnosis apparatus according to claim 1, wherein said electronic module is capable of measuring respiratory air density and molar mass of respiratory air via said second ultrasound transmitter and said second ultrasound receiver.

9. The lung diagnosis apparatus according to claim 1, wherein at least one of said first ultrasound transmitter and said second ultrasound transmitter is also an ultra-sound receiver.

10. The lung diagnosis apparatus according to claim 1, wherein at least one of said first ultrasound transmitter and said second ultrasound transmitter is able to be set to a passive state after deactivation via activation with an alternating voltage that is phase-synchronized and inverse relative to its naturally-occurring resonance oscillation.

11. The lung diagnosis apparatus according to claim 1, wherein said electronic module includes:
means for storing measurements for flow and molar mass; and,
means for calculating an average for all stored values for the flow and the molar mass.

12. The lung diagnosis apparatus according to claim 1, further comprising an additional tube inserted in said respiration tube.
